(19) United States
(12) Patent Application Publication
(10) Pub. No.: US 2003/0109915 A1
(43) Pub. Date: Jun. 12, 2003

(54) Distal protection double balloon catheter

(76) Inventor: T. Anthony Don Michael, Bakersfield, CA (US)

Correspondence Address: BROWDY AND NEIMARK, P.L.L.C., 624 NINTH STREET, NW, SUITE 300, WASHINGTON, DC, 20001-5303, US

(21) Appl. No.: 10/005,699

(22) Filed: Dec. 7, 2001

(51) Int. Cl. A61F 2/06

(52) U.S. Cl. 623/1.11

(57) ABSTRACT

A system for performing a medical treatment in blood vessels, composed of: a catheter having a longitudinal axis, a distal end, an outer lateral surface, a central guidance lumen extending along the longitudinal axis and opening at the distal end, an annular bypass flow lumen surrounding, and isolated from, the guidance lumen, inlet and outlet openings extending from the lateral surface and communicating with the bypass flow lumen, and first and second balloon inflation lumens extending to the lateral surface at respective first and second locations that are spaced apart along the longitudinal axis between the inlet openings and the outlet openings; and first and second balloons secured to the lateral surface and each communicating with a respective one of the first and second inflation lumens, wherein the bypass flow lumen terminates distally at a location between the outlet openings and the distal end of the catheter.

### BACKGROUND OF THE INVENTION

The present invention relates to the treatment of obstructions in body passages, and particularly in arteries.

Treatments of this type typically produce debris that, if allowed to enter the microcirculatory system downstream of the treatment site, can cause damage to organs and tissues.

### BRIEF SUMMARY OF THE INVENTION

The invention provides a novel system that allows an angioplasty treatment, possibly with stenting, to be performed, while preventing the entry of debris resulting from such treatment into the distal microcirculatory system of a patient and assuring a continued supply of blood flow downstream of the obstruction during the angioplasty treatment and protection of organs and tissue downstream of the treatment site against damage that might be caused by debris resulting from the treatment.

The system according to the invention, for performing a medical treatment in blood vessels, is basically composed of: a catheter having a longitudinal axis, a distal end, an outer lateral surface, a central guidance lumen extending along the longitudinal axis and opening at the distal end, an annular bypass flow lumen surrounding, and isolated from, the guidance lumen, inlet and outlet openings extending from the lateral surface and communicating with the bypass flow lumen, and first and second balloon inflation lumens extending to the lateral surface at respective first and second locations that are spaced apart along the longitudinal axis and that are between the inlet openings and the outlet openings; and first and second balloons secured to the lateral surface and each having an interior that communicates with a respective one of the first and second inflation lumens, wherein the bypass flow lumen terminates distally at a location between the outlet openings and the distal end of the catheter.

The annular bypass lumen is formed adjacent the outer wall of the catheter. Therefore, the blood inlet and outlet openings in communication with the bypass flow lumen can be formed in a simple manner. In addition, these openings can be made relative large to assure an adequate blood flow, a flow of at least 30 cc/min being considered necessary to maintain tissue viability.

### BRIEF DESCRIPTION OF THE DRAWING

FIG. 1 is an elevational view of one preferred embodiment of a catheter system according to the invention.

FIGS. 2 and 3 are cross-sectional views taken along lines 2-2 and 3-3, respectively, of FIG. 1.

### DETAILED DESCRIPTION OF THE INVENTION

The following detailed description will be provided with reference to all three Figures.

The system according to the invention is composed essentially of a dilatation and embolic blocking catheter 12 and a surrounding, movable suction catheter 14, which may be in the form of a hypo tube.

Catheter 12 is provided with a central guidewire lumen 20 that is preferably coaxial with the longitudinal axis of catheter 12, a blood bypass flow lumen 22 that surrounds lumen 20 and is separated therefrom by a cylindrical wall 24, a proximal balloon inflation lumen 26 and a distal balloon inflation 28.

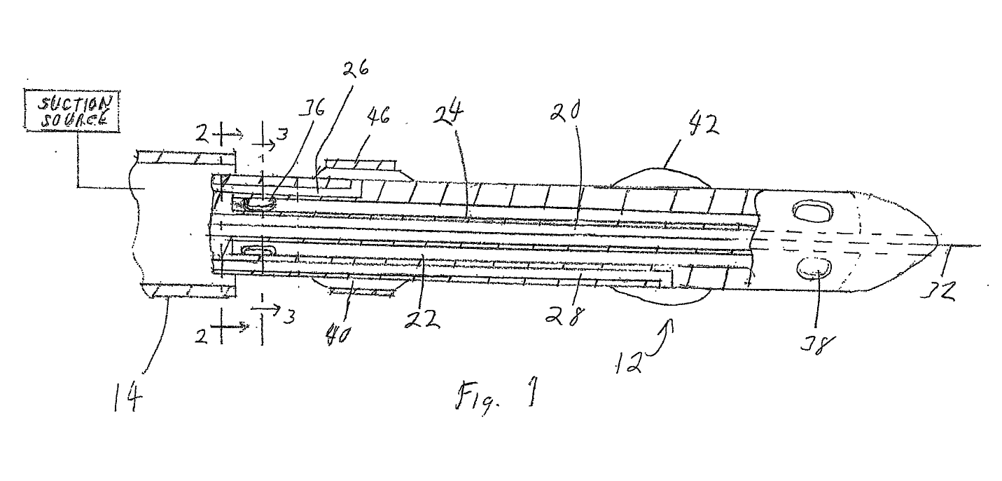

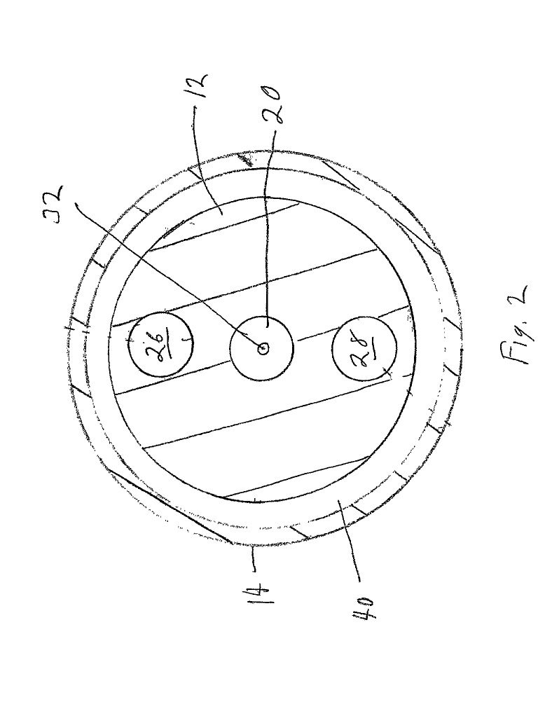

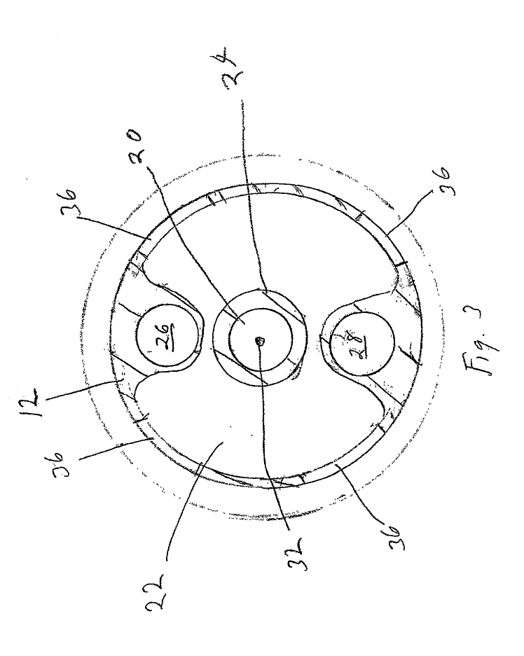

Lumen 20 extends the full length of catheter 12 and in open at the distal end thereof, which is the right-hand end in FIG. 1. Lumen 20 is provided to receive a guidewire 32 that serves to guide catheter 12 to a desired treatment site.

Catheter 12 is provided with a plurality of blood flow inlet openings 36 and a plurality of blood flow outlet openings 38, each set of openings 36, 38 being distributed circumferentially around the outer lateral wall of catheter 12. Openings 36 and 38 extend through the lateral wall of catheter 12 into communication with lumen 22. Lumen 22 does not extend through the full length of catheter 12. The proximal end of lumen 22 extends to a point upstream of openings 36, while the distal end of lumen 22 extends downstream of openings 38. According to the present invention, all openings 36, 38 communicating with lumen 22 extend through the lateral wall of catheter 12. Balloons 40 and 42 are located between openings 36, 38. It is particularly important that the blood flow path defined by lumen 22 extend across balloon 42 because that balloon remains inflated for a longer period of time, of the order of several minutes, than does balloon 40, of the order of a few seconds.

Catheter 12 is completed by two balloons 40 and 42 carried on the outer wall of catheter 12 and each communicating with a respective one of inflation lumens 26 and 28. According to preferred embodiments of the invention, balloon 40 is a low compliance angioplasty balloon, or sheath, and balloon 42 is a high compliance blocking balloon. In further accordance with the invention, balloon 40 carries a stent 46 that is to be expanded and deployed against the inner wall of a body passage to be treated.

Catheter 12 can also be provided with circular radiopaque bands adjacent to the proximal and distal edges of both balloons to assist in proper positioning of the catheter.

In practical embodiments of the invention, catheter 12 can have a size of 2-3F, with a tapered tip, as shown, that helps to allow the catheter to traverse large obstructions.

The above-described device is manipulated to perform an angioplasty treatment in the following manner. Firstly, guidewire 32 is introduced into the blood vessel past the site where a treatment is to be performed. This can be achieved by any conventional procedure that allows guidewire 32 to be advanced through the vessel in the direction of blood flow, i.e. so that the distal end of guidewire 32 points downstream. After the guidewire has been advanced to a point beyond the location of the obstruction to be treated, for example with the aid of radiographic fluoroscopic monitoring, catheter 12 is placed over the guidewire so that the guidewire extends through lumen 20. Catheter 12 is then advanced over the guidewire to the site where the treatment is to be performed, specifically by bringing balloon 40 and stent 46, if provided, to a location opposite the obstruction. Then, tube 14 is inserted in the blood vessel around catheter 12 and brought to a location substantially as shown in FIG. 1, upstream of the treatment site.

Then, balloon 42 is expanded by supplying a fluid at a suitable pressure, usually less that latm, via lumen 28 to block the flow of blood between the outer wall of catheter 12 and the blood vessel wall. After balloon 42 has been thus inflated, blood continues to be supplied to the portion of the blood vessel downstream of catheter 12 by flowing through openings 36, lumen 22 and openings 38.

After balloon 42 has been inflated, balloon 40 is inflated by supplying a fluid at a suitable pressure via lumen 26 to press the obstruction outwardly and to expand and deploy stent 46. This operation generally results in the creation of debris consisting of material that has broken off from the obstruction. This debris will be prevented from flowing downstream of catheter 12 by inflated balloon 42 and will be trapped against the upstream side of balloon 42.

As soon as balloon 40 has been deflated, tube 14 is advanced in the downstream direction toward balloon 42 while suction is applied from an external suction source through tube 14. During this suctioning step, tube 14 can be moved back and forth along the axis of catheter 12 to aid the removal of debris. As a result, debris that has been trapped upstream of balloon 42 will be drawn into tube 14 and removed from the patient's body, where it can be inspected, possibly with the aid of a microscope. After suction has been performed for a sufficient time to assure removal of all debris, or at least all potentially dangerous debris, balloon 42 is deflated and tube 14 and catheter 12 are removed from the blood vessel.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

What is claimed is:

1. A system for performing a medical treatment in a blood vessel while providing downstream microcirculatory system protection, said system comprising:
a catheter having a longitudinal axis, a distal end, an outer lateral surface, a central guidance lumen extending along the longitudinal axis and opening at said distal end, an annular bypass flow lumen surrounding, and isolated from, said guidance lumen, inlet and outlet openings extending from said lateral surface and communicating with said bypass flow lumen, and first and second balloon inflation lumens extending to said lateral surface at respective first and second locations that are spaced apart along the longitudinal axis and that are between said inlet openings and said outlet openings; and
first and second balloons secured to said lateral surface and each having an interior that communicates with a respective one of said first and second inflation lumens,
wherein said bypass flow lumen terminates distally at a location between said outlet openings and said distal end of said catheter.

2. The system of claim 1 further comprising: a tube dimensioned to surround, and to be movable parallel to the longitudinal axis of, said catheter, and to move past at least one of said balloons when that balloon is deflated; and a suction source communicating with the interior of said tube.

3. The system of claim 2 wherein said catheter is tapered at said distal end.

4. The system of claim 2 further comprising an expandable stent mounted or crimped on said first balloon.

5. The system of claim 2 wherein said first balloon is a low compliance angioplasty balloon or sheath and said second balloon is a high compliance blocking balloon and is located between said first balloon said outlet openings.

6. The system of claim 2 wherein there are a plurality of said inlet openings distributed around the longitudinal axis, and a plurality of said outlet openings distributed around the longitudinal axis, and said outlet openings are located between said inlet openings and said distal end.

7. The system of claim 1 wherein said catheter is tapered at said distal end.

8. The system of claim 1 wherein said first balloon is a low compliance angioplasty balloon or sheath and said second balloon is a high compliance blocking balloon and is located between said first balloon said outlet openings.

9. The system of claim 1 wherein there are a plurality of said inlet openings distributed around the longitudinal axis, and a plurality of said outlet openings distributed around the longitudinal axis, and said outlet openings are located between said inlet openings and said distal end.

10. The system of claim 1 further comprising an expandable stent mounted or crimped on said first balloon.
